(19) United States
(12) Patent Application Publication
(10) Pub. No.: US 2008/0214682 A1
(43) Pub. Date: Sep. 4, 2008

(54) METHODS OF TREATING PNEUMONIC PLAGUE

(75) Inventors: Wyndham W. Lathem, St. Louis, MO (US); Virginia Miller, St. Louis, MO (US); William E. Goldman, St. Louis, MO (US)

Correspondence Address: POLSINELLI SHALTON FLANIGAN SUELTHAUS PC, 100 SOUTH FOURTH STREET, SUITE 100, SAINT LOUIS, MO, 63102-1825, US

(73) Assignee: WASHINGTON UNIVERSITY IN ST. LOUIS, St. Louis, MO (US)

(21) Appl. No.: 11/962,293

(22) Filed: Dec. 21, 2007

Related U.S. Application Data
(60) Provisional application No. 60/871,575, filed on Dec. 22, 2006.

(51) Int. Cl. A61K 47/00; A61P 11/00

(52) U.S. Cl. 514/789

(57) ABSTRACT

The present invention relates to methods of treating pneumonic plague. More specifically, the present invention relates to methods of using plasmin or Pla inhibitors to treat pneumonic plague.

### CROSS REFERENCE TO RELATED APPLICATIONS

This application claims the priority of U.S. Provisional Application 60/871,575, filed Dec. 22, 2006, which is hereby incorporated by reference in its entirety.

### GOVERNMENTAL RIGHTS

The present invention was made, at least in part, with support by the National Institutes of Health, National Institute of Allergy and Infectious Diseases grant numbers AI53298 and U54 AI057160. Accordingly, the United States Government may have certain rights in the invention.

### FIELD OF THE INVENTION

The present invention relates to methods of treating pneumonic plague.

### BACKGROUND OF THE INVENTION

Pneumonic plague is the deadliest manifestation of disease caused by the bacterium Yersinia pestis. Although rare compared with the bubonic form of plague, which is acquired by skin penetration, primary pneumonic plague may be transmitted via aerosol droplets, and is highly contagious. The aerosol method of transmission of pneumonic plague may initiate an epidemic of primary pneumonic plague, which if not treated early, is almost always universally fatal. The current worldwide incidence of plague is low by historical standards, but the possible combination of widespread aerosol dissemination and rapid disease progression are of particular concern for defense against bioterrorism.

Y. pestis alone carries pPCP1, a 9.5 kb plasmid that encodes the pesticin, pesticin immunity protein, and the plasminogen activator protease Pla. In models of bubonic plague, the surface protease Pla promotes the invasion of Y. pestis to disseminate from subcutaneous sites of inoculation into the lymphatics and deeper tissues, but is unnecessary to grow at the local site of inoculation. While strains of Y. pestis lacking Pla are reported to be of equivalent or near-equivalent virulence to wild-type by LD₅₀ analysis when introduced via aerosol, the progression of lung and systemic disease has never been evaluated in a model of primary pneumonic plague.

Current treatment for plague comprises antibiotics. To reduce the chance of death, however, antibiotics must be given within 24 hours of the first symptoms. Due to the highly contagious nature of pneumonic plague and the high lethality associated with it, there is a need in the art for more effective treatments.

### SUMMARY OF THE INVENTION

One aspect of the present invention encompasses a method for decreasing the proliferation of Y. pestis in the lungs of a subject infected with Y. pestis. The method comprises inhibiting the protease activity of Pla.

Another aspect of the present invention encompasses a method for decreasing the proliferation of Y. pestis in the lungs of a subject infected with Y. pestis. The method comprises inhibiting the dissolution of fibrin clots in the lungs of the subject.

An additional aspect of the invention encompasses a method for delaying the onset of the pro-inflammatory stage of pneumonic plague in a subject. The method comprises inhibiting Pla protease activity.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

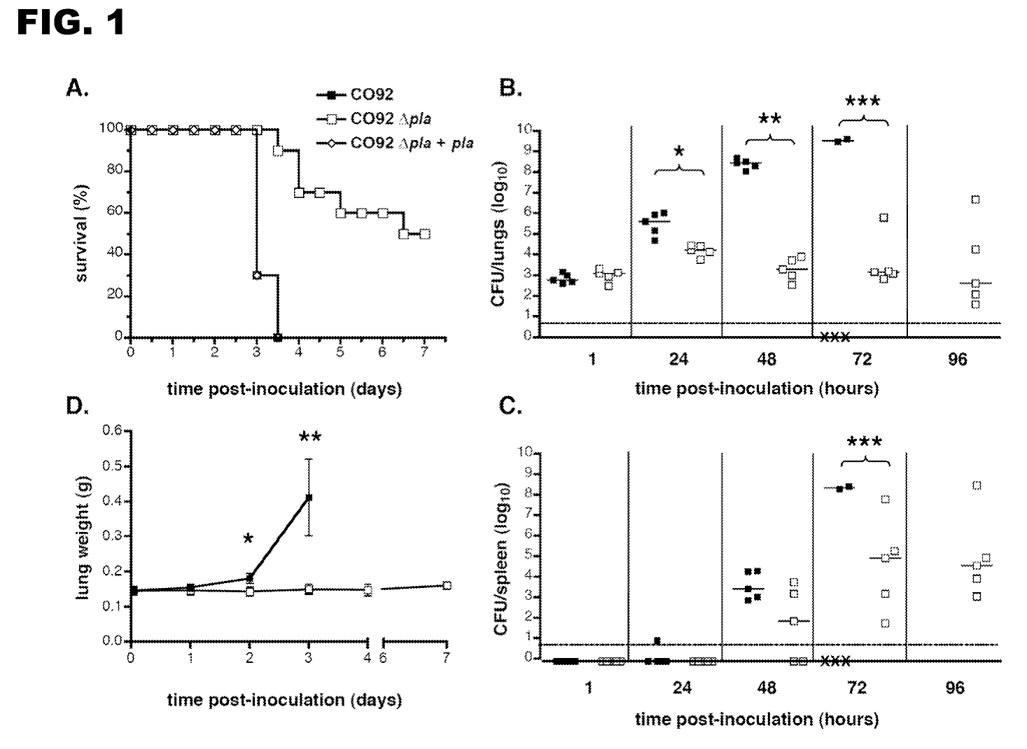

FIG. 1 depicts graphs showing that Pla is required for Y. pestis to cause a primary, acute infection in the lungs of mice. (A) Survival of C57BL/6 mice infected intranasally with Y. pestis CO92 (black squares), Y. pestis CO92 Δpla (white squares), or Y. pestis CO92 Δpla complemented with pla (white diamonds). (B and C) Kinetics of infection with Y. pestis strain CO92 (black squares) or CO92 Δpla (white squares). Bacteria were introduced i.n. to C57BL/6 mice and at various times the CFU per organ in the lungs (B) and spleen (C) were determined. Each point represents the numbers recovered from a single mouse. The limit of detection is indicated by a dashed line. Symbols below the limit of detection represent mice that survived but did not have detectable numbers of bacteria; an “X” indicates a mouse that succumbed to the infection. A solid line indicates the median of CFU recovered. At 24 (*p=0.037), 48, (**p=0.002) and 72 hours (***p<0.001), the CFU of CO92 recovered in the lungs was significantly increased over CO92 Δpla, and at 72 hours (***p<0.001) was significantly higher in the spleen (unpaired t test). (D) Gross weight of lungs from mice infected with Y. pestis CO92 (black squares) or CO92 Δpla (white squares). C57BL/6 mice were infected as above, and at various times, lungs were excised and weighed. At 2 (*p=0.01) and 3 days (**p<0.001), the weights of lungs infected with CO92 were significantly increased over CO92 Δpla, while the weights of lungs infected with CO92 Δpla remained unchanged throughout the course of the infection (unpaired t test). Each experiment was repeated twice.

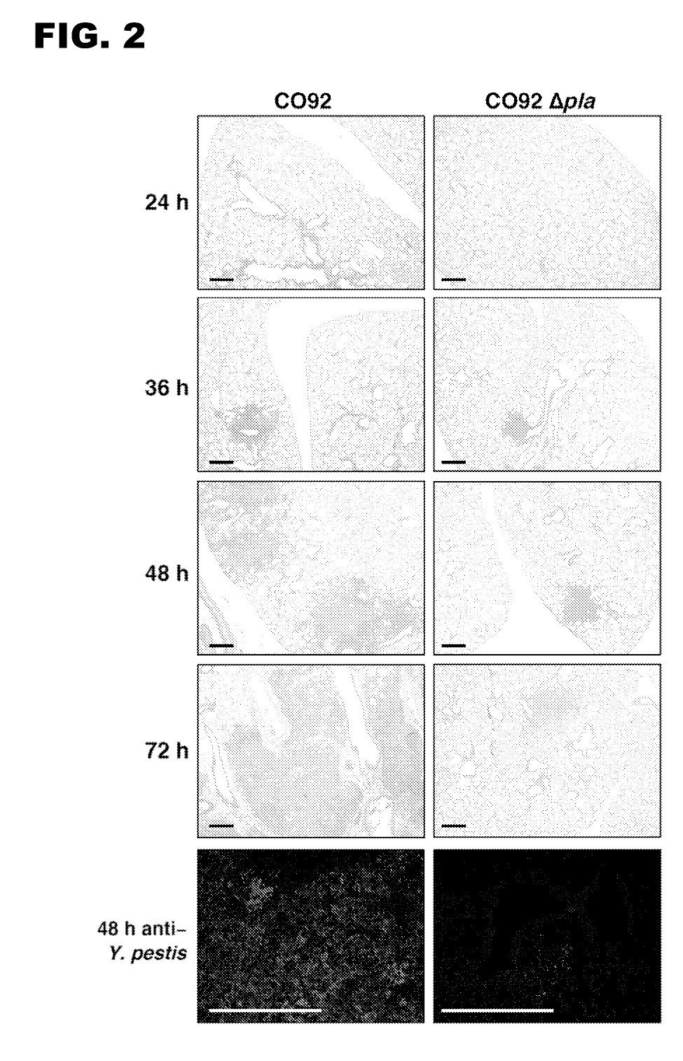

FIG. 2 depicts micrographs showing the histology and presence of bacteria in the lungs of mice infected with Y. pestis CO92 or CO92 Δpla during the progression of pneumonic plague. C57BL/6 mice were infected intranasally and at various times, lungs were inflated and fixed with 10% neutral buffered formalin, embedded in paraffin, and 5-μm sections were stained with hematoxylin and eosin or an anti-Y. pestis antibody. While the number and size of the inflammatory lesions continued to increase in the presence of Y. pestis CO92, they remained relatively restricted and fixed in the presence of CO92 Δpla. In addition, numerous bacilli were detected in the lungs of wild-type infected mice while only few bacteria were visible in the Δpla infection. The panels shown are representative of experiments repeated twice. Bar=200 μm.

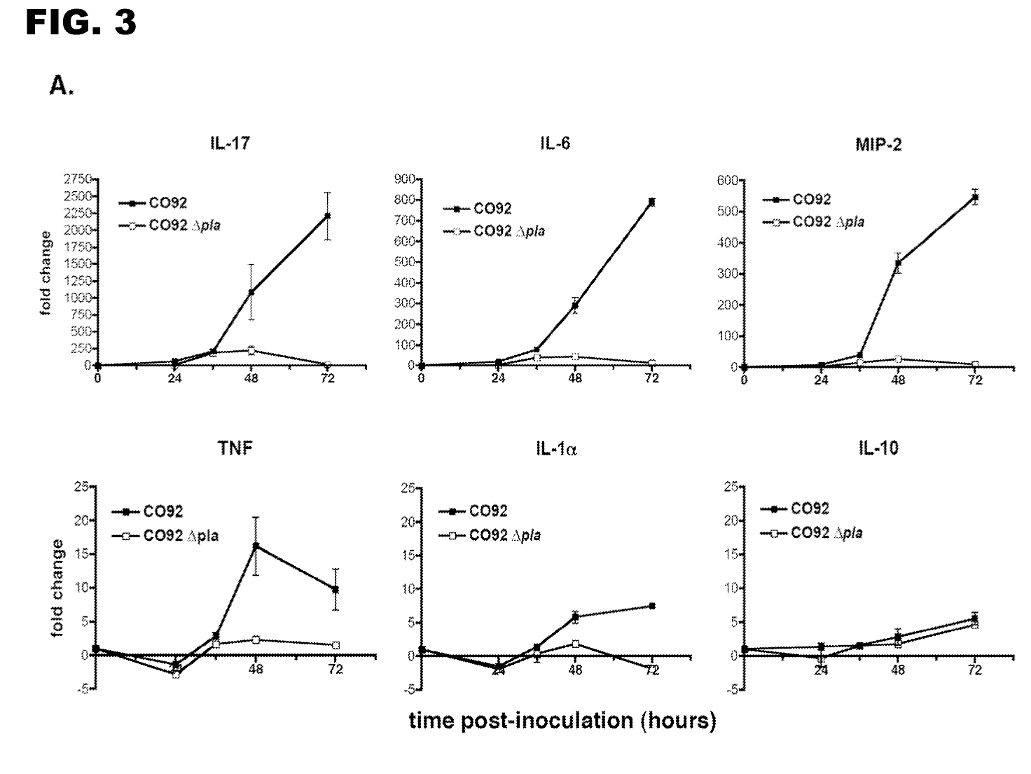

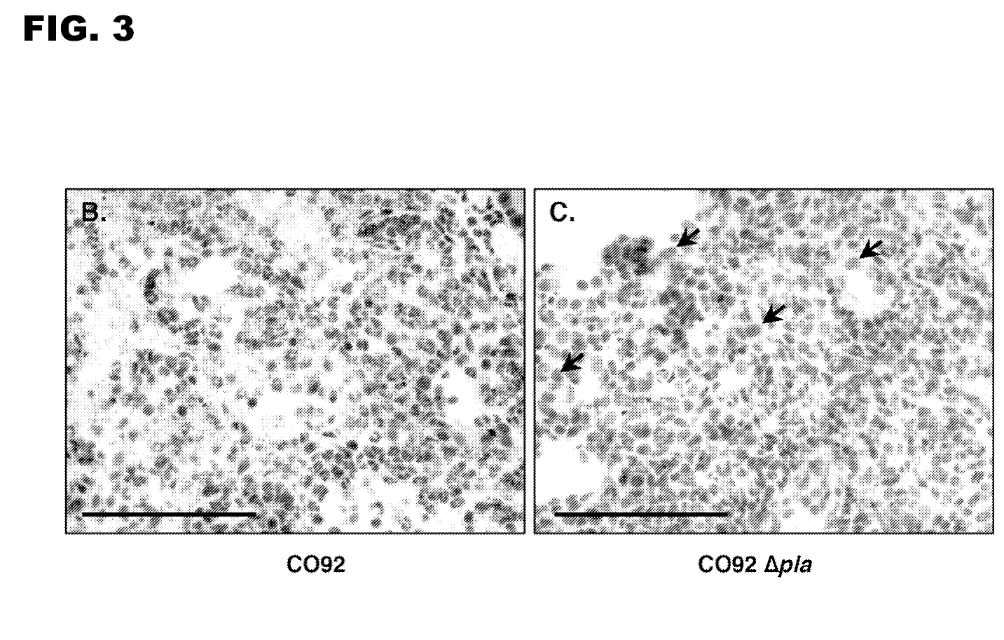

FIG. 3 depicts graphs and micrographs showing the progression of the inflammatory response to Y. pestis CO92 or CO92 Δpla. (A) Mice were uninfected or infected intranasally with bacteria and at various times, lungs were removed and immediately immersed in RNAlater solution. RNA was extracted, reverse-transcribed, and the relative transcript levels of IL-17, IL-6, MIP-2, TNF, IL-1α, and IL-10 compared to uninfected mice were determined by qRT-PCR using the ΔΔC_{T} method (23) and normalized to GAPDH for Y. pestis CO92 (black squares) or CO92 Δpla-infected mice (white squares). Proliferating cell nuclear antigen (PCNA) antibody stain and hematoxylin counter-stain of lungs infected with Y. pestis CO92 (B) or CO92 Δpla (C) after 48 hours. Multiple PCNA-positive cells (arrows) are present in the foci of inflammation in the lungs of Δpla-infected mice but absent in the wild-type infection. Extra-nuclear granular staining in the CO92-infected lungs correlate with the presence of bacteria. Bar=50 μm.

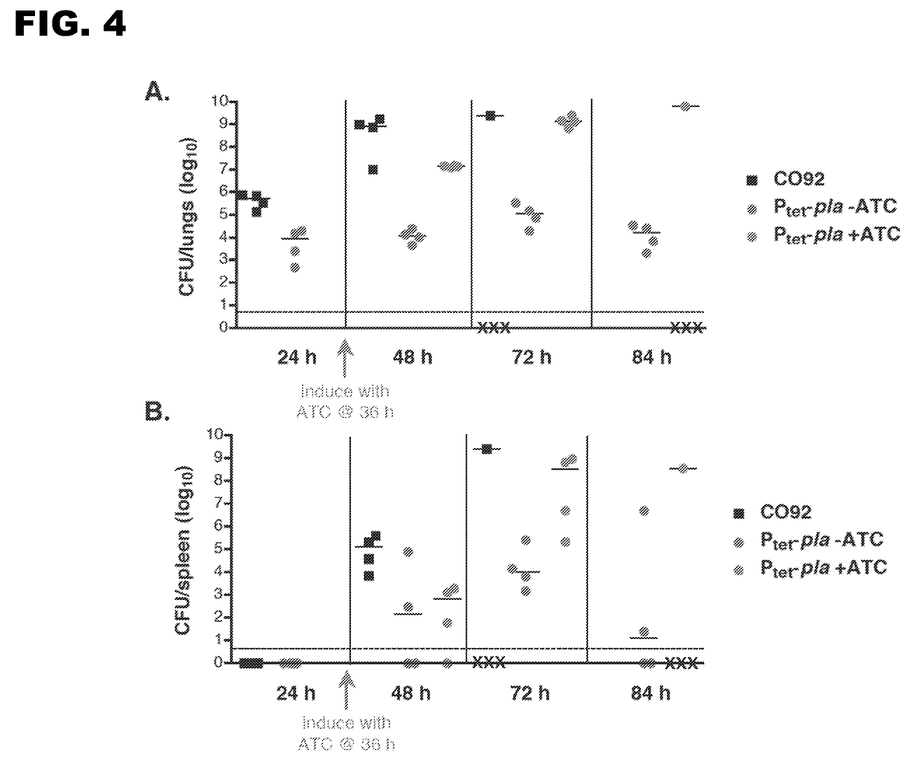

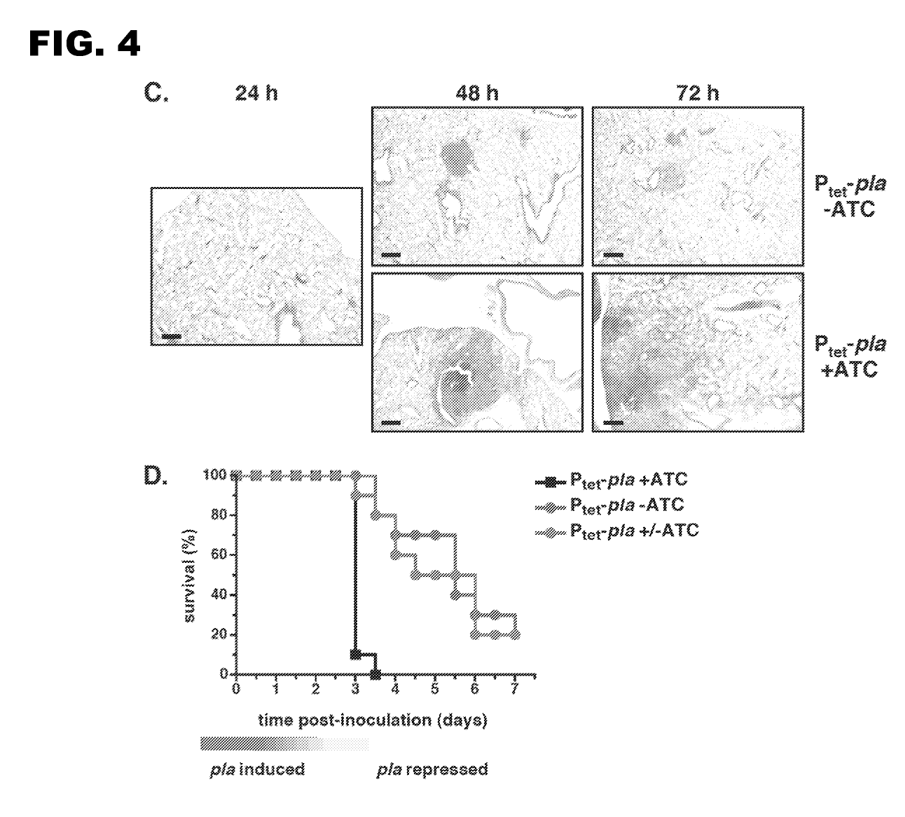

FIG. 4 depicts graphs and micrographs showing the control of primary pneumonic plague progression by the tetracycline-responsive promoter system in Y. pestis. (A and B) Induction of Pla during intranasal infection. Mice were infected i.n. with Y. pestis CO92 (black squares) or CO92 Δpla+Pₜₑₜ-pla prepared in the absence of ATC (repressed state). After 36 hours, mice infected with the Pₜₑₜ-pla strain were administered PBS (blue circles) or ATC (2 mg/kg body weight) (red circles) by i.p. injection bid. CFU in the lungs (A) and spleen (B) were determined at various times. Symbols and lines are as described for FIG. 1. (C) Lung histology of mice infected with the Pₜₑₜ-pla strain and treated with PBS or ATC. Lungs were prepared as described for FIG. 2. Note: there is only one panel at 24 hours post-inoculation as ATC was not administered until 36 hours. Bar=200 μm. (D) Repression of pla expression midway through pneumonic plague infection. Survival of mice infected with CO92 Δpla+Pₜₑₜ-pla in the pla-induced state for the duration of the experiment (black squares)(mean time to death (MTD)=3.1 days), in the pla-repressed state for the duration (blue circles) (MTD=5.1 days), or in the pla-induced state for the first day followed by the pla-repressed state for the remainder of the experiment (red circles) (MTD=4.6 days). See Examples for details. As the turnover rate of Pla on the bacterial cell surface during plague infection and the half-life of ATC in the lungs of mice are unknown, the bar beneath the survival curve approximates the period at which Pla induction ends and repression begins.

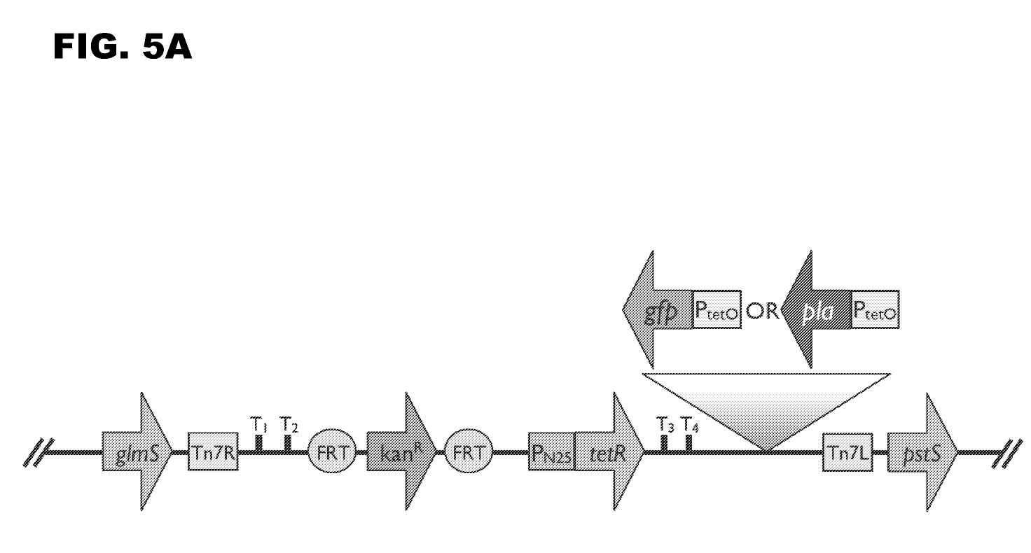

FIG. 5 depicts a diagram and micrographs of constructs used in the invention. (A) Diagram of the Tn7-based transposon insertion contained within the glmS-pstS intergenic region of Y. pestis carrying the tetracycline-responsive system. Strains of Y. pestis were transposed with constructs that include the Tn7L and Tn7R elements, a kanamycin resistance cassette flanked by FRT sites, the tetracycline repressor gene tetR driven by P_{N25}, and either gfp or pla expressed from the Pₜₑₜ promoter. T₁, T₂, T₃, and T₄ indicate transcriptional terminators. (B) GFP fluorescence induced by ATC in Y. pestis during primary pneumonic plague. Mice were infected with strains of Y. pestis carrying P_{N25}-tetR (YP125) or P_{N25}-tetR+Pₜₑₜ-gfp (YP126). PBS (as a placebo) or ATC (2 mg/kg body weight) was given by i.p. injection twice daily to mice infected with YP126, and lungs were examined after 48 hours. GFP fluorescence was visible in plague bacilli treated with ATC, but not PBS. For each fluorescence channel, images were captured with matched exposure times.

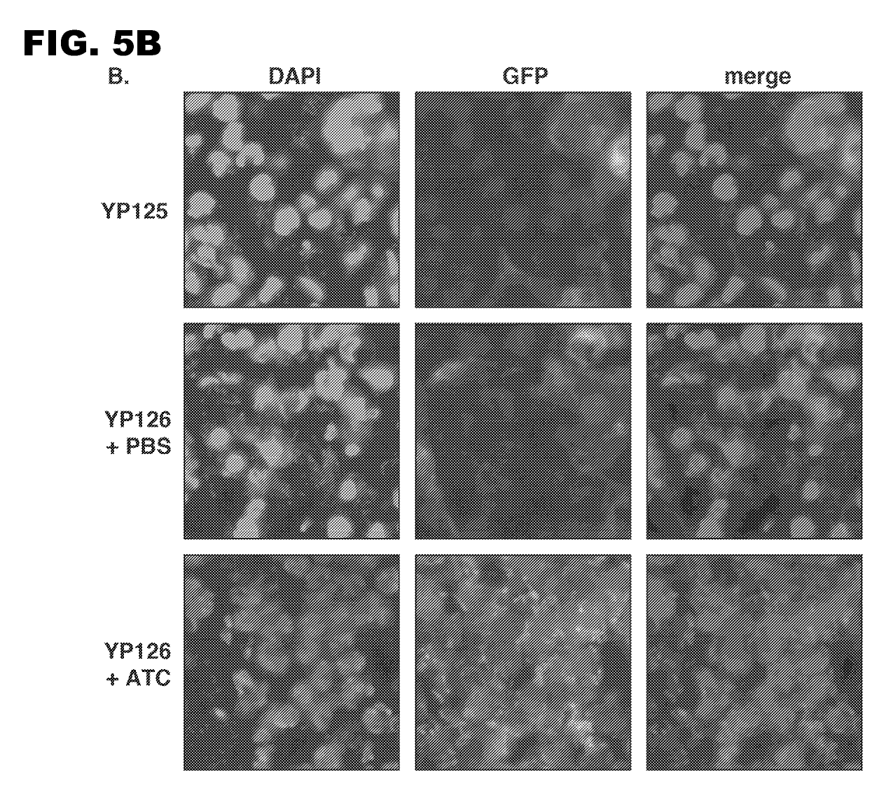

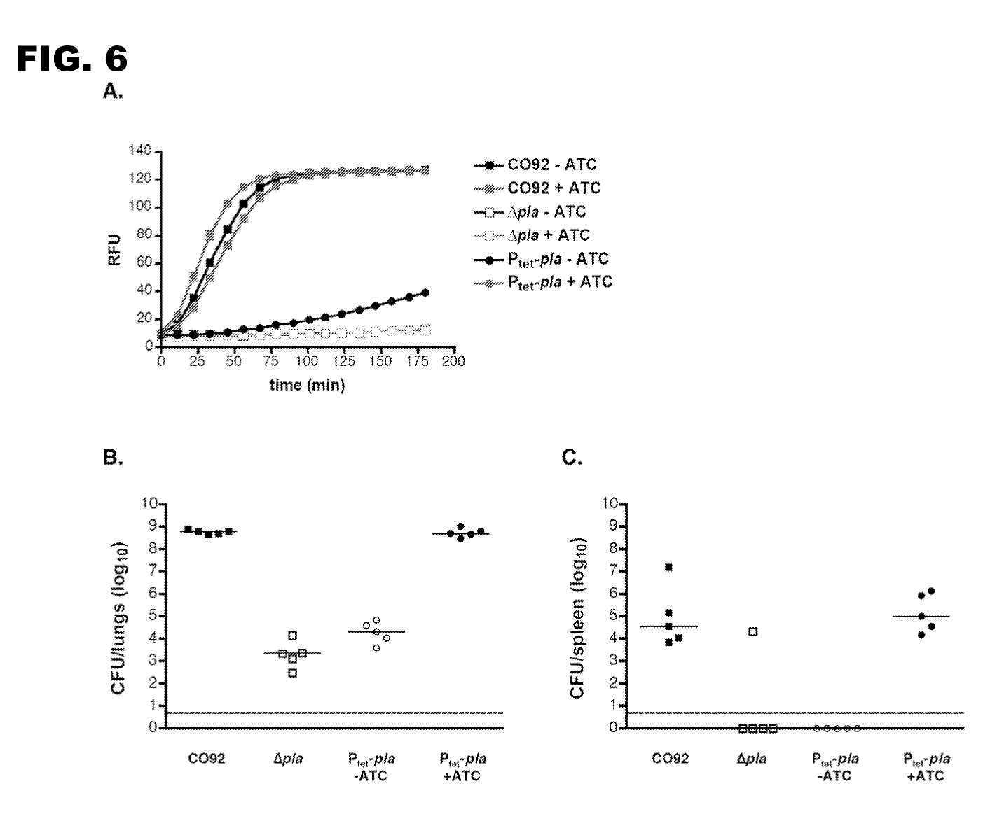

FIG. 6 depicts graphs showing the control of Pla expression and activity by the tetracycline-responsive promoter system in Y. pestis. (A) Plasminogen-activating activity of pCD1⁻Y. pestis CO92 (black squares), CO92 Δpla (white squares), and CO92 Δpla+Pₜₑₜ-pla (circles; YP138 pCD1⁻) in the presence (red) or absence (black) of ATC (0.5 μg/mL). Strains were incubated with human glu-plasminogen and the fluorescent substrate SN-5 in PBS for 3 hours at 37° C. Relative fluorescence was measured every 11 minutes at an excitation wavelength of 360 nm and emission wavelength of 460 nm. In the presence of ATC, CO92 Δpla+Pₜₑₜ-pla exhibits wild-type Pla activity, while the presence or absence of ATC does not affect CO92 Pla activity, nor does the presence of ATC induce plasminogen-activating activity from CO92 Δpla. (B and C) Mice were infected i.n. with 1×10⁴ CFU of Y. pestis CO92 (black squares), CO92 Δpla (white squares), or CO92 Δpla+Pₜₑₜ-pla grown in the presence (black circles) or absence (white circles) of ATC. Mice infected with CO92 Δpla+Pₜₑₜ-pla were administered ATC (2 mg/kg body weight bid) or PBS (as a placebo) by i.p. injection twice daily and CFU in the lungs and spleens after 48 hours were determined. Mice given CO92 Δpla+Pₜₑₜ-pla and treated with ATC were able to fully recapitulate a wild-type infection (no significant difference between CO92 and Pₜₑₜ-pla+ATC), while a similar infection in the absence of ATC approximated the levels of CO92 Δpla (no significant difference between Δpla and Pₜₑₜ-pla−ATC). CFU recovered were significantly different between CO92 and Δpla (p<0.0001), CO92 and Pₜₑₜ-pla−ATC (p<0.001), Δpla and Pₜₑₜ-pla+ATC (p<0.002) and Pₜₑₜ-pla−ATC and Pₜₑₜ-pla+ATC (p<0.002) (unpaired t test). A solid line indicates the median of CFU recovered; a dashed line indicates the limit of detection; “X” indicates mice that succumbed to the infection.

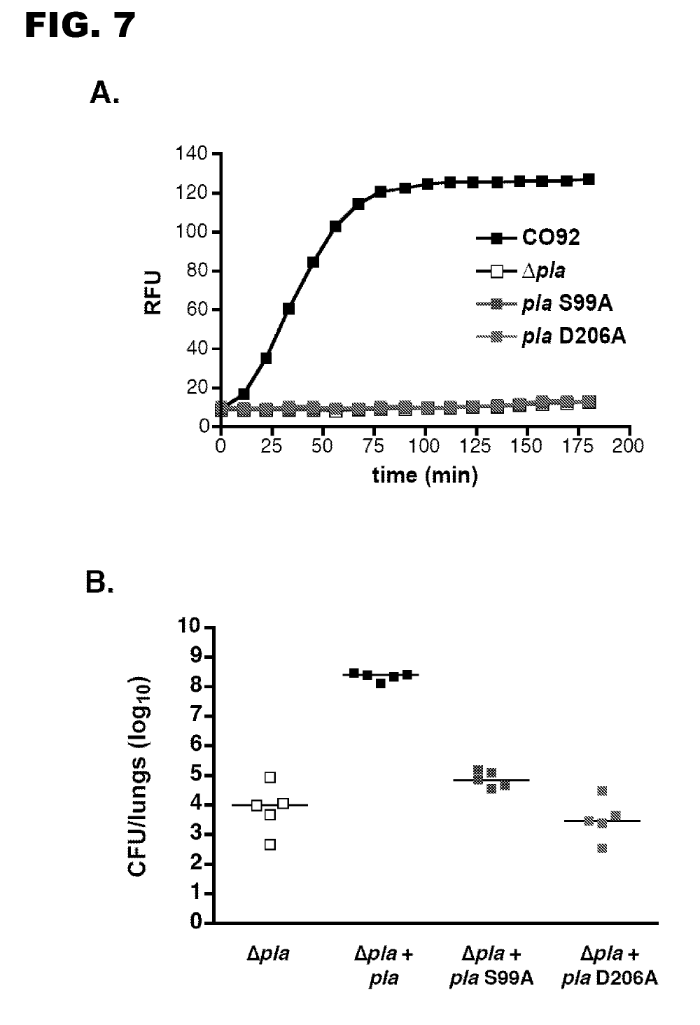

FIG. 7 depicts graphs showing the Plasminogen-activating ability of strains of Y. pestis carrying two independently derived point mutants of Pla. (A) pCD1-isolates of Y. pestis strains CO92 (black squares), CO92 Δpla (white squares), CO92 Δpla+pla S99A (blue squares), and CO92 Δpla+pla D206A (red squares) were incubated with human glu-plasminogen and the fluorescent substrate SN-5 in PBS for 3 hours at 37° C. Relative fluorescence was measured every 11 minutes at an excitation wavelength of 360 nm and emission wavelength of 460 nm. Much like the Δpla derivative and in contrast to the wild-type strain, the two Y. pestis strains carrying independent point mutants of pla are unable to activate plasminogen. (B) Outgrowth of Y. pestis in the lungs requires the plasminogen-activating activity of Pla. Mice were infected i.n. with strains CO92 Δpla (white squares), CO92 Δpla+pla (black squares), CO92 Δpla+pla S99A (blue squares), and CO92 Δpla+pla D206A (red squares). After 48 hours, CFUs in the lungs were enumerated. Neither strain of Y. pestis carrying the proteolytically inactive variants of Pla was able to recapitulate the wild-type infection, demonstrating the requirement of the plasminogen-activating activity of Pla to cause primary pneumonic plague.

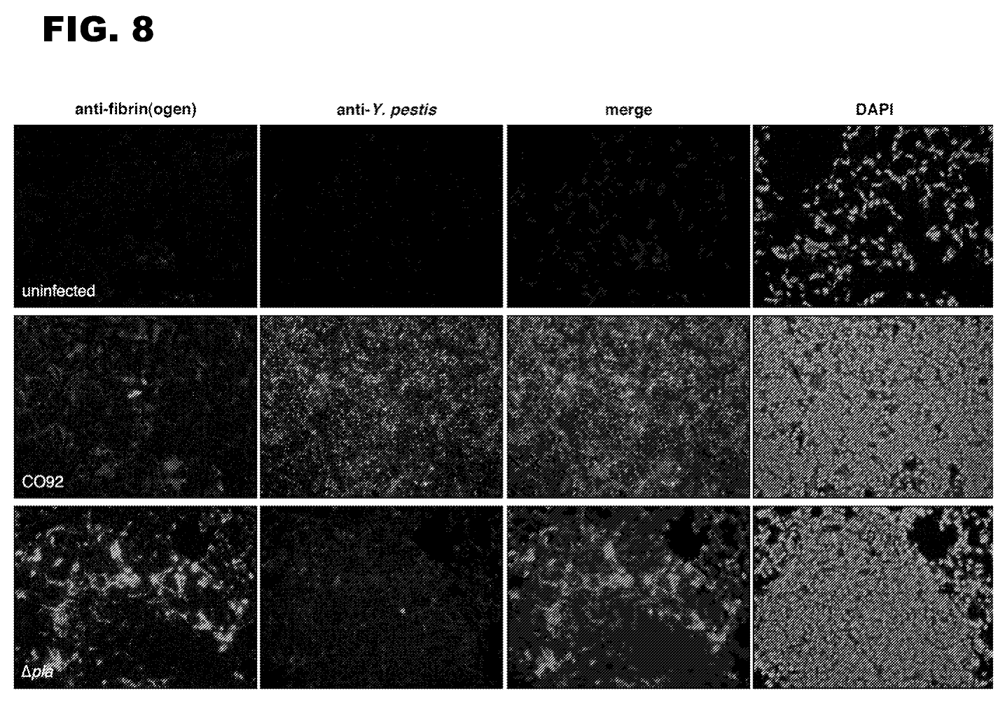

FIG. 8 depicts micrographs showing fibrin(ogen) deposition in the lungs of Y. pestis-infected mice. Mice were uninfected or infected i.n. with strains CO92 or CO92 Δpla for 48 hours. Following fixation, 5 μm lung sections were immunostained with an anti-fibrin(ogen) antibody (red), an anti-Y. pestis antibody (green), and counterstained with DAPI (blue). No fibrin(ogen) staining was detected in the lungs of uninfected mice; in contrast, extensive fibrin(ogen) deposition is evident in the lungs of infected mice. The pattern and extent of fibrin(ogen) staining differs between mice infected with Y. pestis CO92 and CO92 Δpla, however. For each fluorescence channel, images were captured with matched exposure times.

### DETAILED DESCRIPTION OF THE INVENTION

The present invention provides methods of treating pneumonic plaque. In particular, the methods increase the time for which antibiotics may be effectively administered to a subject afflicted with pneumonic plague, and consequently, the methods of the invention may increase the chance of the subject's survival.

### I. Methods of Treating Pneumonic Plaque

In one embodiment, the invention provides methods for delaying the onset of the pro-inflammatory stage of pneumonic plague. Generally speaking, the pro-inflammatory stage of pneumonic plague begins approximately 24 to approximately 40 hours after infection, and is characterized by an increase in inflammatory cytokine production in the subject. Non-limiting examples of inflammatory cytokines that increase in the pro-inflammatory stage include IL-17, IL-6, MIP-2, TNF, and IL-1α. As used herein, “delaying the onset” of the pro-inflammatory stage refers to delaying the onset of inflammatory cytokine production by about 4, 8, 12, 16, 20, 24, 28, 32, 36, or more hours. In some embodiments, the onset of the pro-inflammatory stage may be delayed by one or more days. In an exemplary embodiment, the pro-inflammatory stage may be delayed long enough to administer antibiotics to the subject.

In another embodiment, the method comprises decreasing the proliferation of Yersinia pestis in the lungs of the subject. Generally speaking, decreasing the proliferation of Yersinia pestis in the lungs will reduce the severity of the pneumonia in the subject. Additionally, decreasing the proliferation of Yersinia pestis in the lungs will further reduce the chances the subject will infect other individuals via aerosolized respiratory droplets comprising Y. pestis.

Typically, the methods for treating pneumonic plague may be utilized after a subject is infected with Y. pestis but before the subject dies of the infection. In some embodiments, the methods may be utilized within the first 96 hours, 84 hours, 72 hours, 60 hours, 48 hours, 36 hours, or 24 hours after infection. In exemplary embodiments, the methods may be utilized before the pro-inflammatory stage of pneumatic plague.

### A. Serine Protease Inhibitors

One aspect of the invention encompasses treating a subject with pneumonic plague by administering a serine protease inhibitor. Generally speaking, administering a serine protease inhibitor increases the time for which antibiotics may be effectively administered in a subject afflicted with pneumonic plague, delays the onset of the pro-inflammatory stage of pneumonic plague, and/or decreases the proliferation of Yersinia pestis in the lungs of the subject. In an exemplary embodiment, the serine protease inhibitor inhibits the dissolution of fibrin clots, in particular, fibrin clots in the lungs.

In some embodiments, the serine protease inhibitor may have antifibrinolytic activity. Serine protease inhibitors that have antifibrinolytic activity are well known in the art. Non-limiting examples include aprotinin, aminocaproic acid, and tranexamic acid. In other embodiments, the serine protease inhibitor may be capable of inhibiting the serine protease plasmin. In one embodiment, the serine protease inhibitor may specifically inhibit plasmin. In another embodiment, the serine protease inhibitor may non-specifically inhibit plasmin.

In other embodiments, the serine protease inhibitor is capable of inhibiting serine proteases that activate plasminogen, thereby forming plasmin. Non-limiting examples of serine proteases that activate plasminogen include tissue plasminogen activator (tPA) and plasminogen activator (Pla).

Assays to determine whether a compound is a serine protease inhibitor are well known in the art, including assays to determine whether a serine protease inhibitor has antifibrinolytic activity. For instance, the clot lysis assay may be used. Briefly, plasma may be mixed with small amounts of fibrin and treated to facilitate clotting. Plasmin and a serine protease inhibitor may be incubated with the clot, and the percent lysis determined. In some methods, the percent lysis may be determined by measuring the release of labeled, soluble fibrin peptide.

### B. Plasmin Activity Inhibitors

The invention also encompasses a method of treating pneumonic plague comprising inhibiting plasmin activity. In some embodiments, plasmin activity may be inhibited by administering a compound that inhibits plasmin activity. Generally speaking, inhibiting plasmin activity increases the time for which antibiotics may be effectively administered in a subject afflicted with pneumonic plague, delays the onset of the pro-inflammatory stage of pneumonic plague, and/or decreases the proliferation of Yersinia pestis in the lungs of the subject. In an exemplary embodiment, inhibiting plasmin activity inhibits the dissolution of fibrin clots, in particular, fibrin clots in the lungs.

As used herein, “plasmin activity” may refer to plasmin serine protease activity, plasminogen translation, plasminogen transcription, plasmin protein concentration and plasminogen mRNA concentration. Inhibiting plasmin activity may encompass, in part, inhibiting plasmin serine protease activity, inhibiting plasminogen transcription, and inhibiting plasminogen translation. Inhibiting plasmin activity may also encompass, in part, increasing the degradation of plasmin protein and/or mRNA, or decreasing the activation of plasminogen to form plasmin. In each of the above embodiments of the invention, plasmin activity may be specifically inhibited, or, alternatively, plasmin activity may be non-specifically inhibited.

Inhibitors of plasmin activity are known in the art. Non-limiting examples of plasmin activity inhibitors include aprotinin, tissue factor pathway inhibitor-2, aminocaproic acid, and tranexamic acid. In some embodiments, the inhibitor of plasmin activity comprises a KD1 domain, or alternatively, a modified KD1 domain such that the inhibitor has greater specificity for plasmin than other serum serine proteases. Inhibiting plasmin activity may include inhibiting the activation of plasminogen, decreasing the degradation of plasmin proteases, such as α₂-antiplasmin, or inhibiting Pla activity.

Assays to determine the inhibition of plasmin activity are known in the art. For instance, an in vitro clot lysis assay may be performed. Generally speaking, such an assay consists of measuring the amount of time needed for clot dissolution in a sample comprised of the test compound, compared to a control sample that did not receive the test compound. Alternatively, the inhibition of plasmin activity may be assayed in vivo using methods known in the art.

### C. Pla Activity Inhibitors

An additional aspect of the invention encompasses treating a subject with pneumonic plague, the method comprising inhibiting Pla activity. As used herein, “Pla” refers to the plasminogen activator protease encoded by the pPCP1 plasmid of Yersinia Pestis. Generally speaking, inhibiting Pla activity increases the time for which antibiotics may be effectively administered in a subject afflicted with pneumonic plague, delays the onset of the pro-inflammatory stage of pneumonic plague, and/or decreases the proliferation of Yersinia pestis in the lungs of the subject. In some embodiments, Pla activity may be inhibited by administered a compound that inhibits Pla activity. In other embodiments, the compound that inhibits Pla activity may inhibit the dissolution of fibrin clots, in particular, fibrin clots in the lungs.

As used herein, “Pla activity” may refer to Pla protease activity, Pla translation, Pla transcription, Pla protein concentration, and Pla mRNA concentration. Inhibiting Pla activity may encompass, in part, inhibiting Pla protease activity, inhibiting Pla transcription, and inhibiting Pla translation. Inhibiting Pla activity may also encompass, in part, increasing the degradation of Pla protein and/or mRNA. In each of the above embodiments of the invention, Pla activity may be specifically inhibited, or, alternatively, Pla activity may be non-specifically inhibited.

In one embodiment, the Pla protease activity inhibited is Pla plasminogen-activating activity. Pla plasminogen-activating activity may be inhibited, for instance, by blocking the active catalytic nucleophile of Pla, or blocking the proposed substrate recognition loop L4 of Pla, as detailed in the examples. In another embodiment, Pla α₂-antiplasmin degradation activity may be inhibited. In yet another embodiment, Pla activity may be inhibited by a serine protease inhibitor. In still another embodiment Pla activity may be inhibited by a small molecule or a peptide. For instance, by way of non-limiting examples, the peptides identified in Agarkov et al, Bioorg. Med. Chem. Lett. (2007), doi:10.1016/j.bmcl.2007.09.104, or variations thereof, may be used.

Assays to determine the inhibition of Pla activity are known in the art, and are detailed in the Examples. For instance, the assay detailed in Agarkov et al, Bioorg. Med. Chem. Lett. (2007), doi:10.1016/j.bmcl.2007.09.104, hereby incorporated by reference in its entirety, may be used.

### D. Combination Therapy

A method of the invention may further comprise administering an antibiotic to the subject. For instance, in some embodiments, in addition to inhibiting Pla activity, inhibiting plasmin activity, and/or administering a serine protease inhibitor, the subject may also be treated with antibiotics. The antibiotics may be administered before, simultaneously, or after inhibition of Pla activity, inhibition of plasmin activity, and/or administration of a serine protease inhibitor. Antibiotics are well known in the art, and include aminoglycosides, carbacephems, carbapenems, cephalosporins, glycopeptides, macrolides, monobactams, penicillins, polypeptides, quinolones, sulfonamides, tetracyclines, and others. In one embodiment, the antibiotic is selected from the group of antibiotics comprising streptomycin, gentamicin, a tetracycline, and chloramphenicol. The dosage and route of administration will vary depending upon the patient and the antibiotic used, and may be determined by those skilled in the art. For instance, see Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493. In some embodiments, streptomycin, 1 gm IM twice daily for 10 days, or gentamicin, 5 mg/kg IM or IV once daily for 10 days, or chloramphenicol, 25 mg/kg IV 4 times daily for 10 days may be administered to adults.

### E. Compounds, Dosages, and Routes of Administration

The serine protease inhibitors, plasmin activity inhibitors, and Pla activity inhibitors described above may exist in tautomeric, geometric or stereoisomeric forms. The present invention contemplates all such compounds, including cis- and trans-geometric isomers, E- and Z-geometric isomers, R- and S-enantiomers, diastereomers, d-isomers, I-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures of R and S forms for each stereocenter present.

In a further embodiment, the inhibitors of the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine- (N-methylglucamine) and

Solid dosage forms for oral administration may include capsules, tablets, pills, powders, and granules. In such solid dosage forms, the compound is ordinarily combined with one or more adjuvants appropriate to the indicated route of administration. If administered per os, the compound can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

The amount of the compound of the invention that may be combined with the carrier materials to produce a single dosage of the composition will vary depending upon the patient and the particular mode of administration. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Generally speaking, an inhibitor of the invention may be administered after infection of the subject with Y. pestis, but before the death of the subject. In some embodiments, an inhibitor may be administered within the first 96 hours, 84 hours, 72 hours, 60 hours, 48 hours, 36 hours, or 24 hours after infection. In exemplary embodiments, an inhibitor of the invention may be administered before the pro-inflammatory stage of pneumatic plague.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### DEFINITIONS

As used herein, “subject” refers to a mammal capable of being infected with Yersinia Pestis. Subjects may include laboratory animals, such as mice, rats, or guinea pigs. Alternatively, subjects may include humans.

As used herein, “treating” is used in its broadest sense to mean affecting disease progression. In an exemplary embodiment, treating means slowing disease progression in a subject, compared to an untreated subject. For instance, in some embodiments, treating may refer to delaying the onset of the pro-inflammatory stage of pneumonic plague. In other embodiments, treating may refer to decreasing the proliferation of Yersinia pestis in the lungs of the subject.

### EXAMPLES

The following examples illustrate various iterations of the invention.

### Materials and Methods for Examples 1-7

Reagents, bacterial strains, and growth conditions. All chemicals were obtained from Sigma Chemical Company (Saint Louis, Mo.) unless otherwise indicated. A list of bacterial strains, plasmids, and oligonucleotides is described in Table A. The virulent, wild-type Y. pestis strain CO92 was obtained previously from the U.S. Army, Fort Detrick, Md. The presence of pCD1, pMT1, pPCP1, and the pgm locus were confirmed by PCR. Y. pestis was routinely grown on brain-heart infusion (BHIi) agar (Difco) at 26° C. for 2-3 days. Avirulent strains lacking the pCD1 plasmid were isolated by repeated passage at 37° C. on BHI plates containing magnesium oxalate and confirmed by PCR. For liquid cultures, Y. pestis was grown in BHI broth at 26° C. for 6-8 hours in a roller drum before being diluted to an O.D.₆₂₀ of 0.05-0.1 in 10 ml BHI broth with 2.5 mM CaCl₂ in a 125-ml Erlenmeyer flask. Unless otherwise indicated, bacteria were incubated at 37° C. in a water bath shaker set at 250 revolutions per minute for 16-18 hours. For subcutaneous infections of animals, bacteria were prepared at 26° C. in BHI broth on a platform shaker set at 250 revolutions per minute for 16-18 hours.

TABLE A

| | | |
|---|---|---|
| Strains | Genotype/relevant feature | Source/reference |

| | | |
|---|---|---|
| Y. pestis CO92 | pCDI⁺ pMT1⁺ pPCP1⁺ pgm⁺ | Laboratory stock |
| YP102 | Y. pestis CO92 Δpla | This study |
| YP111 | YP102 complemented with pla on pPCP1 | This study |
| YP30 | CO92 carrying pWL204 | This study |
| YP125 | CO92 carrying P_{N25}-tetR integrated | This study |
| | in the glmS-pstS intergenic region | |
| YP126 | CO92 carrying P_{N25}-tetR + Pₜₑₜ-gfp | This study |
| | integrated in the glmS-pstS | |
| | intergenic region | |
| YP135 | YP102 carrying pla S99A on pPCP1 | This study |
| YP136 | YP102 carrying pla D206A on pPCP1 | This study |
| YP138 | YP102 carrying P_{N25}-tetR + Pₜₑₜ-pla | This study |
| | integrated in the glmS-pstS | |
| | intergenic region | |
| YP138pCD1 | pCD1-cured (avirulent) | This study |
| | derivative of YP138 | |

| | | |
|---|---|---|
| Plasmids | Relevant feature | Source/reference |

| | | |
|---|---|---|
| PWL204 | pKD46 (1) derivative | This study |
| | carrying sacB for | |
| | sucrose counterselection: | |
| | amp^{R} | |
| PUC18R6Kmini- | Mini-Tn7 transposition vector; | This study |
| Tn7T-Kan | amp^{R} kan^{R} | |
| pWL212 | pUC18R6K-mini-Tn7T-Kan carrying | This study |
| | P_{N25}-tetR: amp^{R} kan^{R} | |
| pWL213 | pUC18R6K-mini-Tn7T-Kan carrying | This study |
| | P_{N25}-tetR + Pₜₑₜ-gfp: | |
| | amp^{R} kan^{R} | |
| pWL214 | pUC18R6K-mini-Tn7T-Kan carrying | This study |
| | P_{N25}-tetR + Pₜₑₜ-pla: | |
| | amp^{R} kan^{R} | |
| pZS4 | Source for P_{N25}-tetR | (17) |
| pLP-PROTet- | Source for Pₜₑₜ | Clontech |
| 6xHN | | |
| pTNS2 | Plasmid carrying TnsABC + D | (26) |
| | specific transposition | |
| | pathway: amp^{R} | |
| pROBE-gfp | Source for gfp and terminators | (28) |
| | T₃ and T₄: kan^{R} | |
| pLH29 | Plasmid carrying IPTG-inducible | (29) |
| | FLP recombinase, cm^{R} | |

| | | |
|---|---|---|
| Oligonucleotides | Sequence | Source/reference |

| | | |
|---|---|---|
| pla 5′-500 | CGCCTGCTGGCTGCACTTGTCGTTG | This study |
| | (SEQ ID NO:1) | |
| P1 pla 3′-3 | GAAGCAGCTCCAGCCTACACCATTA | This study |
| | GACACCCTTAATCTCTCTGCATG | |
| | AAC | |
| | (SEQ ID NO:2) | |
| p4 pla 5′938 | GGTCGACGGATCCCCGGAATGAAAA | This study |
| | ATACAGATCATATCTCTCTTTTC | |
| | ATC | |
| | (SEQ ID NO:3) | |
| pla 3′ + 500 | CTGGAGAGCAAGTAATGAGAACA | This study |
| | TTA | |
| | (SEQ ID NO:4) | |
| P1 pla 3′-939 | GAAGCAGCTCCAGCCTACACTCAGA | This study |
| | AGCGATATTGCAGACCCGCCG | |
| | (SEQ ID NO:5) | |
| Tet1 | GGCCCTTTCGTCTTCACCTC | (30) |
| | (SEQ ID NO:6) | |
| Tet2 | TAGCTCCTGA AAATCTCGCC | (30) |
| | (SEQ ID NO:7) | |
| P_{N25}-tetR Sac5′ | GGGAGCTCTTAGCGCGAATTGTCGA | This study |
| | GGG | |
| | (SEQ ID NO:8) | |
| Term 2 Bam 3′ | CGGGATCCCCTGGCAGTTTATGGCG | This study |
| | GGCG | |
| | (SEQ ID NO:9) | |
| Pₜₑₜ-405 5′Pst | AACTGCAGCTTTCACCAGCGTTTCT | This study |
| | GGGTG | |
| | (SEQ ID NO:10) | |
| Pₜₑₜ-1 3′ | GGGTACCTTTCTCCTCTTTAATG | This study |
| | (SEQ ID NO:11) | |
| Pₜₑₜ + gfp 5′ 1 | CATTAAAGAGGAGAAAGGTACCC | This study |
| | ATGAGTAAAGGAGAAGAACTT | |
| | TTC | |
| | (SEQ ID NO:12) | |
| gfp BAM 3′725 | CGGGATCCTTATTTGTATAGTTCAT | This study |
| | CCATGCC | |
| | (SEQ ID NO:13) | |
| Pₜₑₜ-pla 5′1 | CATTAAAGAGGAGAAAGGTACCCAT | This study |
| | GAAGAAAAGTTCTATTGTGGC | |
| | (SEQ ID NO:14) | |
| pla 3′939 Sma | CCCCCGGGTCAGAAGCGATATTGCA | This study |
| | GACC | |
| | (SEQ ID NO:15) | |
| pla 3′ + 127 | GCGCCCCGTCATTATGGTGAAAAAG | This study |
| | (SEQ ID NO:16) | |
| pla 5′ S99A | ACAGATCACGCATCTCATCCTGC | This study |
| | TAC | |
| | (SEQ ID NO:17) | |
| pla 3′ S99A | GTAGCAGGATGAGATGCGTGATCT | This study |
| | GTC | |
| | (SEQ ID NO:18) | |
| pla 5′ D206A | GCACATGATAATGCTGAGCACTAT | This study |
| | ATG | |
| | (SEQ ID NO:19) | |
| plal 3′ D206A | CATATAGTGCTCAGCATTATCATG | This study |
| | TGC | |
| | (SEQ ID NO:20) | |

procaine. All of these salts may be prepared by conventional means from the corresponding compound by reacting, for example, the appropriate acid or base with the any of the compounds of the invention.

The inhibitors of the present invention may be formulated into pharmaceutical compositions and administered by a number of different means that will deliver a therapeutically effective dose. Such compositions may be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Deletion and complementation of pla. A deletion of the gene encoding Pla was constructed by a modified form of lambda red recombination originally described by Datsenko and Wanner (24). Briefly, 500 bp upstream and 500 bp downstream of pla were independently amplified by PCR with the oligonucleotides pla 5′-500 and P1 pla 3′3 (upstream region), and P4 pla 5′938 and pla 3′+500 (downstream region). The resulting products were gel-purified and combined with a Kan^{R} cassette flanked by FRT sites (previously amplified by PCR from the plasmid pKD13 (24)) in a second PCR amplification using pla 5′−500 and pla 3′+500. YP30, a strain of Y. pestis CO92 carrying pWL204, a derivative of pKD46 (24) containing the red recombinase genes and the levansucrase gene sacB (for sucrose counterselection), was grown at 26° C. in the presence of 10 mM arabinose (to induce the recombinase genes) and transformed with the gel-purified pla-FRT-Kan^{R}-FRT-pla PCR product. Recombinants were selected on BHI plates containing kanamycin (50 μg/ml). pWL204 was cured from recombinants by passage on BHI plates containing 5% sucrose. The Kan^{R} cassette introduced in the previous step was resolved by the introduction of pLH29, a plasmid carrying the FLP recombinase gene under the control of the lac promoter, and growth overnight at 26° C. in the presence of IPTG (1 mM). Kan^{S}, Cm^{s} recombinants (indicating the loss of pLH29) were identified and confirmed by PCR to create Y. pestis CO92 Δpla. Due to the multi-copy nature of pPCP1 (25), a complementing clone of pla was constructed in the Δpla strain in its original locus by lambda red recombination in a similar manner. Full length, wildtype pla, including 500 bp upstream of the translational start site, was amplified by PCR from Y. pestis strain CO92 with the oligonucleotides pla 5′-500 and P1 pla 3′939, gel-purified and then PCR-amplified in a second reaction with the Kan^{R} cassette and the 500 bp downstream region of pla before being introduced into the Δpla strain carrying pWL204. Recombinants were selected as described above and confirmed by PCR to contain only the pPCP1 plasmid carrying the restored, complementing clone. The Kan^{R} cassette was subsequently excised as described above.

Construction of proteolytically inactive pla S99A and pla D206A strains. The CO92 pla S99A and pla D206A clones were created using the PCR-based method of overlap extension before being introduced into the Δpla strain carrying pWL204, as described above. The first two PCR amplifications used primer (i) pla 5′-500 and its partner mutagenic primer pla 3′S99A or pla 3′D206A, and (ii) pla 3′+127 and its partner mutagenic primer pla 5′S99A or pla 5′D206A. The next PCR amplification contained the resulting products and the primers pla 5′-500 and P1 pla 3′ 938; strains were then constructed as described for the wild-type pla complementation. The constructed Y. pestis CO92 Δpla+pla S99A strain is designated YP135; Y. pestis CO92 Δpla+pla D206A is designated YP136.

Construction of strains carrying ATC-inducible genes. Strains carrying anhydrotetracycline (ATC)-inducible genes were constructed in Y. pestis by using Tn7-based integration of the genes into the chromosomal glmS-pstS intergenic region as follows: pWL212, the base Tn7 plasmid carrying the tetR gene driven by the constitutive P_{N25} promoter followed by two transcriptional terminators, was generated by PCR amplifying P_{N25}-tetR from pZS4 with the primers Tet1 and Tet2, splicing by overlap extension (SOE)-PCR to T₃ and T₄ from pROBE-gfp with the primers P_{N25}/tetR Sac 5′ and Term 4 Bam 3′, and cloning the product into pUC18R6K-mini-Tn7T-Kan (26). Into this base system the genes for either gfp or pla, controlled by the Pₜₑₜ promoter, were added. The Pₜₑₜ promoter plus additional sequence was amplified from pLP-PROTet-6xHN with the primers Pₜₑₜ-405 5′ Pst and Pₜₑₜ-1 3′. The genes for gfp, from pROBE-gfp, or pla, from Y. pestis CO92, were amplified with the primer pairs Pₜₑₜ+gfp 5′1 and gfp Bam 3′725 or Pₜₑₜ+pla 5′1 and pla 3′ 939 Sma, respectively. The resulting products were joined to the Pₜₑₜ promoter by SOE-PCR and cloned into pWL212 to create pWL213 or pWL214, respectively. pWL212, pWL213, or pWL214 were individually electroporated along with pTNS2 (26), a plasmid carrying the TnsABC+D specific transposition pathway, into either wild-type Y. pestis CO92 or the equivalent Δpla strain and transformants were selected on BHI plates containing kanamycin. The Kan^{R} cassette was then resolved via the introduction of pLH29 as described above and Kan^{S}, Cm^{s} recombinants were identified and confirmed by PCR to create YP125 (Y. pestis CO92 P_{N25}-tetR), YP126 (Y. pestis CO92 Pₜₑₜ-gfp), or YP138 (Y. pestis CO92 Δpla Pₜₑₜ-pla). The Tn7-based integrons were confirmed by PCR and are outlined in FIG. 5A.

Animals. All animal experiments were approved by the Washington University Animal Studies Committee, protocols #20050189 and #20060154. Pathogen-free 6-8 week-old female C57BL/6 mice were obtained from the Jackson Laboratory (Bar Harbor, Me.) and were housed in high efficiency particulate air-filtered barrier units kept inside biological safety cabinets for the duration of the experiments. Mice were given food and water ad libitum and were kept at 25° C. with alternating 12-hour periods of light and dark. Bacteria were grown in BHI broth as described above, washed once in sterile phosphate-buffered saline (PBS), and maintained at 37° C. (for i.n. infections) or room temperature (for s.c. infections). Mice were lightly anesthetized and inoculated by the intranasal route with 20 μL of Y. pestis in PBS or by subcutaneous injection with 50 μl of Y. pestis in PBS. Actual numbers of colony-forming units (CFU) inoculated were determined by plating serial dilutions onto BHI agar. Animals that were clearly moribund or on the verge of death were humanely euthanized with an overdose of pentobarbital sodium (150 mg/kg).

Kinetics and survival curves. Groups of 10 mice were infected intranasally with 1×10⁴ CFU of Y. pestis CO92, CO92 Δpla, or CO92 Δpla+pla. Mice were monitored twice daily for 7 days, and any surviving mice were euthanized. For experiments examining the kinetics of infection, groups of 4-5 mice were infected intranasally with 1×10⁴ CFU or subcutaneously with 150 CFU of Y. pestis. At various times post-infection, mice were sacrificed, the lungs and spleens surgically removed, weighed, and homogenized in 0.5 ml sterile PBS, and serial dilutions were plated onto BHI agar. Results are reported as CFU/organ. In experiments involving anhydrotetracycline (ATC) induction of bacterial gene expression, PBS or ATC (2 mg/kg diluted in PBS) was administered by i.p. injection twice daily as indicated. For the survival curve in which pla was induced then subsequently repressed (FIG. 4D), Y. pestis strain YP138 was cultured either in the presence or absence of ATC (0.5 μg/ml) at 37° C. for 16-18 hours. Ten mice were infected intranasally with YP138-ATC and 20 mice infected with YP138+ATC. Of the latter 20 mice, 10 were given ATC (2 mg/kg diluted in PBS) by i.p. injection twice daily for the duration of the experiment; the remaining 10 mice were given ATC at the time of infection and after 12 hours, thus promoting pla expression for at least the first day of the infection. Mice were monitored twice daily for 7 days, and any surviving mice were euthanized.

Histopathology, immunohistochemistry, and immunofluorescence. Groups of 3-5 mice were infected intranasally with 1×10⁴ CFU of Y. pestis. Uninfected mice and mice infected for 24, 36, 48, or 72 hours were sacrificed with an overdose of pentobarbital sodium (150 mg/kg) and their lungs inflated with 10% neutral buffered formalin via cannulation of the trachea. Lungs were removed and fixed in 10% formalin overnight before being embedded in paraffin. Five-μm sections of tissue were stained either with hematoxylin and eosin, immunostained with an anti-Y. pestis antibody, an antibody against the proliferating cell nuclear antigen (PCNA) (Santa Cruz Biotechnology, Santa Cruz, Calif.), or an anti-fibrin(ogen) antibody (DakoCytomation, Denmark) using standard procedures before being examined.

Cytokine analysis. Groups of 3 mice were infected intranasally with 1×10⁴ CFU of Y. pestis CO92 or CO92 Δpla. Uninfected mice and mice infected for 24, 36, 48, or 72 hours were sacrificed with an overdose of pentobarbital sodium (150 mg/kg) and the lungs removed and immediately submerged in an excess of RNAlater RNA stabilization solution (Ambion, Woodward, Tex.). Total RNA was purified from lung tissue with the RiboPure RNA extraction kit (Ambion), treated with DNase, and reverse-transcribed with a set of random primers and the SuperScript II polymerase (Invitrogen, Carlsbad, Calif.) in triplicate according to the manufacturers' instructions. cDNAs were used as templates for amplification and detection of the mouse genes IL-17, TNF, IL-6, MIP-2, IL-1α, and IL-10 with the SYBR Green dye (Bio-Rad, Hercules, Calif.) in an iCycler thermocycler (Bio-Rad). For each gene, the calculated threshold cycle (Cₜ) was normalized to the Cₜ of the GAPDH gene from the same cDNA sample before calculating the fold change using the ΔΔCₜ method (23).

Plasminogen activation assay. Strains were grown for 6 hours at 26° C. before being diluted to 8×10⁷ CFU in PBS and combined with purified human glu-plasminogen (Hematologic Technologies, Essex Junction, Vt.) (4 μg) and the chromogenic substrate D-AFK-ANSNHiC₄H₉-2HBr (SN-5; Hematologic) (50 μM) in a total volume of 200 μl of PBS as described previously (18). Reaction mixtures were incubated in triplicate for 3 hours at 37° C., and the absorbance at 460 nm was measured every 11 minutes in a Synergy HT microplate reader.

### Example 1

C57BL/6 mice were infected intranasally with wild-type Y. pestis CO92, an isogenic Y. pestis strain lacking Pla (CO92 Δpla), or the Δpla strain complemented with coding sequence of Pla. Mice given wild-type Y. pestis CO92, a strain isolated from a fatal case of pneumonic plague (12), succumbed to the infection in a highly synchronous manner. In contrast, by 7 days post-inoculation, only 50% of the mice infected with the Δpla strain developed terminal plague, and the rate at which the mice died was significantly less synchronous than those infected with the wild-type strain (FIG. 1A). Complementation of the mutant with the coding sequence for Pla fully restored virulence to wild-type levels. Thus, the lack of Pla delayed the time to death and affected the synchronous nature of the disease in these mice. This delay led to the assessment of the bacterial load in the lungs and spleen (CFU/organ) at various times post-inoculation. As previously observed (13), the kinetics of bacterial growth during infection with the wild-type strain proceeded consistently and rapidly, approaching 10¹⁰ CFU in the lungs (FIG. 1B) and 10⁹ CFU in the spleen (FIG. 1C) by 72 hours. The kinetics of bacterial growth in the Δpla-infected mice were significantly altered, however. After 24 hours, approximately 10³-10⁴ bacteria were recovered from the lungs of Δpla-infected mice, levels 100-1000-fold lower than that recovered from wild-type-infected mice at the same point (FIG. 1B).

Over the next two days, the numbers of Δpla bacteria in the lungs did not significantly change, while wild-type bacteria increased by almost 6 logs in the lungs over the same period. Although there was little change in the bacterial load in the lungs, bacteria were detected in the spleens of 3/5 mice after 48 hours and all mice by 72 hours, indicating that escape of the Δpla strain from the lungs to distal organs can still occur (FIG. 1C). Indeed, at both 72 and 96 hours, one of five mice had a bacterial burden approaching 10⁸-10⁹ CFU in the spleen. This corresponded with increased numbers of bacteria in the lungs, however this is likely due to the recirculation of plague bacilli from the blood back into the lungs (13) rather than an outgrowth of bacteria in this organ.

These data demonstrate that Pla controls the proliferation of Y. pestis in the lungs but is expendable for the bacteria to fully disseminate systemically to distal tissues. Remarkably, these results are distinct from the phenotype attributed to Pla in models of bubonic plague: when Pla⁻ Y. pestis is introduced subcutaneously, dissemination is dramatically reduced but bacterial outgrowth at the local site of infection is unaffected (8, 9). Indeed, rates of dissemination from the initial site of colonization to the spleen are substantially increased when bacteria are introduced intranasally compared to the subcutaneous route (Table B). Table B shows the percentage of mice that had detectable Y. pestis CFUs in the spleen following intranasal or subcutaneious infection with the wild-type strain CO92 of the isogenic strain CO92 Δpla after 24, 48, 72, or 96 hours, of the total number of surviving mice infected. The ability of Y. pestis lacking Pla to escape the respiratory tract may be attributed to the highly vascularized nature of the lung, in that the escape of a few bacilli into an alveolar capillary may be sufficient to initiate a systemic infection.

TABLE B
Rates of Y. Pestis dissemination to the spleen

| | |
|---|---|
| Intranasal | Subcutaneous |

| | CO92 | CO92 Δpla | CO92 | CO92 Δpla |
|---|---|---|---|---|
| 24 h | 10% | 0% | 0% | 0% |
| 48 h | 100% | 50% | 40% | 0% |
| 72 h | 100% | 90% | 100% | 40% |
| 96 h | NS* | 100% | 100% | 40% |

*NS = no survivors

### Example 2

A hallmark of fatal bacterial pneumonia is the accumulation of edematous fluid in the lungs due to the disruption of cell-cell junctions in the bronchi and alveoli, allowing plasma leakage into the airspace. This accumulation can be measured by a change in gross lung weight, as the fluid and cells contribute to an increased mass of the organ. By 48 hours post-inoculation, mouse lungs infected with wild-type Y. pestis weighed significantly more than uninfected lungs, and by 72 hours were 3-4-fold heavier (FIG. 1D), indicating a severe pneumonia that is most likely the cause of death. Interestingly, the lungs of mice infected with the Δpla strain of Y. pestis showed no change in gross weight, even by 7 days, suggesting that the death of mice infected with this strain is not due to a fatal pneumonia but rather more likely caused by a systemic infection. Indeed, the appearance and proliferation of bacteria in the spleens is consistent with this observation. The results, therefore, may explain the similar LD₅₀ values between the wildtype and Pla- strains when inhaled, even though Y. pestis requires Pla to cause a severe pneumonia.

Conflicting characterizations of the mammalian host response to subcutaneous infection with strains of Y. pestis lacking Pla show either an increased accumulation (8) or equivalent levels (9) of inflammatory cells at the site of infection compared to the fully virulent strain. Previous work has demonstrated that primary infection of the mouse lung results in an overwhelming inflammatory infiltrate consisting mainly of neutrophils with a significant rise in multiple cytokines, chemokines, and other proinflammatory molecules (13). To reconcile these observations with the lack of bacterial outgrowth and fluid accumulation in the lungs of Δpla-infected mice, lung sections were examined of wild-type and mutant Y. pestis infected mice stained with hematoxylin and eosin. An influx of inflammatory cells was detected in the lungs in both the wild-type and Δpla infections as early as 36 hours after inoculation; in both cases, the predominant infiltrating cell type was polymorphonuclear (FIG. 2). Whereas the size of the pulmonary lesions in the wild-type infection proceeded to increase over time, resulting in tissue destruction and hemorrhage, the foci of inflammation in mice infected with CO92 Δpla remained relatively constant and restricted. To correlate the presence of bacteria with the inflammatory lesions, infected lung sections were examined using immunofluorescence with an anti-Y. pestis antibody (a kind gift from J. Hinnebusch). While numerous extracellular bacilli in and around areas of inflammation in the lungs of wild-type-infected mice were observed, only a few bacteria restricted to the inflammatory lesions were detected in the Δpla infection (FIG. 2). Thus, the development of a severe respiratory infection during primary pneumonic plague is directly attributable to the expression of Pla, and both bacterial outgrowth and the subsequent inflammatory response in the lungs are dependent on this surface protease.

### Example 3

Previous studies on the progression of pneumonic plague revealed an early anti-inflammatory stage that is maintained until the relatively sudden onset of a pro-inflammatory phase, beginning at approximately 36 hours post-inoculation (13). The lack of bacterial outgrowth and the presence of a restricted inflammatory infiltrate in response to Pla⁻ Y. pestis in the lungs of mice suggest that the host immune state is able to control the pulmonary infection while preventing what otherwise becomes an overwhelming inflammatory reaction to the bacteria. With this in mind, the level of immune activation in the lungs was assessed using quantitative reverse-transcription PCR (qRT-PCR) to determine changes in transcript levels of multiple pro- and anti-inflammatory mediators. Consistent with earlier observations (13), mice infected with Y. pestis CO92 remained unresponsive to infection in the first 24-36 hours, with the majority of cytokines relatively unchanged compared to uninfected mice (FIG. 3A). By 48 hours, however, most cytokines were dramatically upregulated, and these trends continued into the terminal phase of plague at 72 hours. Also consistent with previous reports for Yersinia infections (14, 15), IL-10 transcript levels showed little change throughout the entire course of infection. Similarly, at 24 and 36 hours the cytokine transcript levels in the lungs of mice infected with Δpla were also relatively unchanged compared to uninfected (and wild-type-infected) mice, demonstrating that the anti-inflammatory stage of the disease remains relatively consistent between these two strains. At 48 hours post-inoculation, however, cytokine transcript levels in response to Δpla were unchanged or only slightly increased, a striking contrast to the wild-type infected mice. By the following day of infection, transcript levels for all cytokines except IL-10 decreased, suggesting control over and downregulation of the inflammatory response to Y. pestis Δpla. Thus, these data suggest that in the absence of Pla, an anti-inflammatory state is maintained in the lungs but the infection is unable to progress to the pro-inflammatory phase. Furthermore, the Pla-dependent transition to this second phase appears to be critical for the severe pneumonia that results during pneumonic plague, suggesting that Pla may act as a “molecular gatekeeper” for the events in the latter stages of the disease.

That cytokine transcript levels appeared to stabilize and then decrease during infection suggested a possible resolution of the pulmonary inflammatory lesions in the Δpla-infected mice. Wild-type and Δpla-infected lungs were immunostained for the proliferating cell nuclear antigen (PCNA), a marker for host cellular DNA synthesis (16). While the host cells of wild-type-infected lungs were almost uniformly PCNA-negative (FIG. 3B), large numbers of PCNA-positive cells were present in Δpla-infected mice (FIG. 3C), indicating active cell proliferation and regeneration of the tissue in the lungs of these animals. Lung repair at this stage of the infection is further evidence that fatalities among Δpla-infected mice are not a consequence of airway inflammation or damage, but instead are the result of systemic spread of the organisms to extrapulmonary sites.

### Example 4

The deletion of pla from the plague bacillus converts a rapidly progressing pneumonic infection into a non-pneumonic disease. If Pla alone acts to control the ability of Y. pestis to cause pneumonic plague, it was hypothesized that experimental induction of pla expression mid-way during the aborted pulmonary disease would be sufficient to turn the non-pneumonic infection into a pneumonic one. To test this hypothesis, the tetracycline-responsive promoter system was adapted to exogenously control gene expression in Y. pestis during infection. This system uses a transcriptional modulator, a tetracycline-responsive promoter, and an antibiotic of the tetracycline family, in this case anhydrotetracycline (ATC), to induce or repress gene activity (17).

To test the ability of Pla to specifically control the development of a rapidly progressing pneumonia by the plague bacillus, the Δpla strain of Y. pestis carrying pla under the control of the tetracycline-responsive promoter (Y. pestis CO92 Δpla Pₜₑₜ-pla, strain YP138) in the absence of ATC (pla-repressing conditions) and administered bacteria to mice intranasally. After 36 hours, normally the mid-point of the pneumonic infection with a wild-type strain, pla expression was induced by providing ATC to mice by intraperitoneal injection and then followed the progression of the infection by assessing the bacterial load and pathology of the lungs. Bacteria in the pla-repressed state established a non-progressive lung infection in a manner similar to the Δpla-infected mice, further demonstrating the requirement for Pla by Y. pestis in this organ. However, once ATC is administered and Pla expression is upregulated, the condition of these mice quickly converts to a disease with all the features of pneumonic plague (FIG. 4, A and B): rapid proliferation of bacteria with development of visible microcolonies, unrestricted inflammatory infiltrate, tissue damage, and shortened time to death (FIG. 4C). Thus, the absence of Pla stalls the development of disease in the early anti-inflammatory phase but does not eliminate the potential of these organisms to cause pneumonic plague. Ultimately, the block in the progression of infection by the respiratory route, including the lack of lung damage and inflammation, is completely reversible by the expression of Pla, thereby demonstrating its control over the disease.

Pla may facilitate the invasive nature of Y. pestis by converting host plasminogen into plasmin while degrading the plasmin inhibitor α₂-antiplasmin, thereby releasing bacteria from the entrapment of fibrin clots (18, 19). Indeed, recent evidence has shown that fibrin deposition is an important means of immune control of a variety of pathogens (20-22), and thus the subversion of the coagulation cascade may be a significant virulence mechanism during infection. Consistent with this hypothesis, data show that the plasminogen-activating activity of Pla is essential to Y. pestis virulence in the pulmonary system (see FIG. 7, A and B). Fibrin(ogen) deposition can be detected in the lungs of mice infected with either the wild-type or Δpla strain, but the pattern and extent of fibrin(ogen) immunostaining is substantially altered (FIG. 8). The role of Pla during pneumonic plague may help explain why Y. pestis has acquired the ability to cause a rapid and severe respiratory infection and be transmitted from person to person by the aerosol route, while the closely related Y. pseudotuberculosis and Y. enterocolitica, the other pathogenic species of Yersinia, do not.

### Example 5

The critical role for Pla suggests that its inhibition may offer a therapeutic advantage, particularly since the rapid progression of primary pneumonic plague leaves little time for effective treatment once symptoms become apparent. This was tested experimentally by infecting mice with the Pla inducible Y. pestis strain YP138 prepared in the presence of ATC and provided ATC to the animals for only the first day of the infection, allowing the ATC to be cleared and pla expression to be repressed for the remainder of the experiment. As seen in FIG. 4D, the time until death is clearly delayed when the Pla-induced state is switched to a Pla-repressed state during the infection; in fact, the kinetics more closely resemble those of a Δpla infection. This suggests that exogenous inhibition of Pla during primary pneumonic plague may indeed prolong the survival of the affected individual, expanding the time frame during which antibiotics could be successfully administered to treat the disease.

### Example 6

To adapt the tetracycline-responsive promoter system for use in Y. pestis during primary pneumonic plague infection of mice, the sensitivity of Y. pestis to anhydrotetracycline (ATC), a less-toxic analog of tetracycline with increased binding affinity to the tetR product (27) was tested. It was found that the bacterium was resistant up to at least 2 μg/mL, well in excess of the maximal ATC-based induction of the system. By using the Tn7 site-specific transposon system (26), the tetR gene expressed from the constitutive P_{N25} promoter in single copy was integrated onto the chromosome of Y. pestis. To confirm that the presence of TetR itself does not alter the virulence of Y. pestis during pneumonic plague, the kinetics of infection of the Tn7-tetR-integrated Y. pestis strain (designated YP125) was analyzed using the mouse model of infection described previously (13). The virulence of the bacterium is unaffected by this integration. The gene for the green fluorescent protein (GFP) was cloned, driven by the tetracycline-responsive promoter (Pₜₑₜ), into the P_{N25}-tetR-containing construct downstream of the tetR gene. The construct was then integrated in single copy onto the chromosome of Y. pestis, resulting in the insertion outlined in FIG. 5.

To determine if GFP expression in Y. pestis could be induced in the lungs of mice during pneumonic plague, mice were infected intranasally with Y. pestis strain YP125 (containing only the tetR gene, as a negative control) or GFP strain YP126 prepared in vitro in an uninduced state (i.e. no ATC provided). Then, to mice infected with YP126, PBS (as a placebo) or ATC (2 mg/kg body weight) was administered every 12 hours by i.p. injection. After 48 hours, Y. pestis could be easily detected in the lungs of all mice, however, only bacteria in mice treated with ATC were GFP-positive; GFP fluorescence was not detected from YP125 and PBS-treated YP126 in this infection (FIG. 5B).

Then strain YP138 was created, in which a construct carrying tetR and pla was integrated onto the chromosome of Y. pestis CO92 Δpla, to modulate the expression of the virulence factor Pla (FIG. 5). The ability to control Pla activity was determined by incubating a pCD1- (avirulent) version of YP138 grown in the presence or absence of ATC with purified human plasminogen and SN-5, a fluorescent substrate of plasmin. When induced with ATC, YP138 pCD1- was able to achieve wild-type levels of plasminogen activation, while in the absence of ATC, only minimal levels of plasmin activity were detected (FIG. 6A). The ability of YP138 induced with ATC to recapitulate a wild-type infection in the lungs was tested. YP138 was grown in the presence or absence of ATC and then administered to mice intranasally. Mice were then treated with PBS or ATC as described above, and after 48 hours, the bacterial burden in the lungs was evaluated. Strain YP138 treated with ATC reached wild-type levels in the lungs, while the numbers of placebo-treated YP138 bacteria were slightly higher but not significantly different than that of CO92 Δpla (likely due to the minimal Pla activity the strain produces under non-inducing conditions, as observed in FIG. 6A). Thus, these data demonstrate that the tetracycline-responsive system of gene expression is able to control the virulence of Y. pestis during primary pneumonic plague (FIG. 6, B and C).

### Example 7

As a bacterial cell surface-associated protein, the primary function of the Y. pestis Pla during mammalian infection is thought to be its ability to alter the coagulation and fibrinolytic cascades by proteolytically activating the plasmin precursor plasminogen while simultaneously inactivating the plasmin inhibitor α2-antiplasmin (18, 8), thereby altering the immune response during infection and preventing the entrapment of bacteria in fibrin clots. While the ability of Pla to act on these targets has been demonstrated in vitro, it has not been determined whether the plasminogen-activating activity of Pla is required in vivo, particularly in the lung. To assess this function of Pla during primary pneumonic plague infection of mice, two independent point mutations that abolish the plasminogen-activating activity of the enzyme were made (18): a serine to alanine at residue 99 (pla S99A) and an aspartic acid to alanine at reside 206 (pla D206A). Serine 99 is hypothesized to serve as the active catalytic nucleophile of Pla (18), while aspartic acid 206 is contained within the proposed substrate recognition loop L4 of the protein (18). These mutant forms of pla were reintegrated onto pPCP1 in Y. pestis CO92 Δpla. To confirm that these mutants were proteolytically inactive, CO92, CO92 Δpla, CO92 pla S99A, and CO92 pla D206A were incubated with purified human glu-plasminogen and a fluorescent substrate of plasmin, SN-5. While the wild-type strain exhibited abundant plasminogen-activating ability, the Δpla, pla S99A, and pla D206A strains were unable to activate plasminogen during the course of the assay, confirming that the mutants were enzymatically inactive (FIG. 7A). C57BL/6 mice were infected intranasally with the Δpla strain, strains containing the two Pla point mutants, or the Δpla strain complemented with a wild-type copy of pla that is isogenic to the point mutants (Δpla+pla) and is able to restore full virulence to the Δpla strain (FIG. 1A). After 48 hours, the bacterial load in the lungs was assessed. Mice infected with CO92 Δpla and CO92 Δpla+pla had bacterial counts of approximately 10⁴ CFU and 10⁸ CFU, respectively (FIG. 7B), while mice infected with either pla S99A or D206A had dramatically decreased bacterial counts compared to mice infected with the pla complemented strain (approximately 10⁴-10⁵ CFU and 10⁴ CFU, respectively). Thus, these data demonstrate that the protease activity of Pla is essential to Y. pestis virulence in the pulmonary system.

The fact that the plasminogen-activating ability of Pla is required for Y. pestis to cause primary pneumonic plague led to the assessment of the deposition of fibrin(ogen) in the lungs of wild-type and Δpla-infected mice. Sections of lungs from uninfected mice or mice infected for 48 hours with CO92 or CO92 Δpla were stained with a polyclonal antibody that reacts with fibrinogen, fibrin, and the fibrinogen fragments D and E, co-stained an anti-Y. pestis antibody, and counterstained with DAPI. While there was no evidence of fibrin(ogen) deposition in the lungs of uninfected mice, abundant fibrin(ogen) staining was observed in and around foci of inflammation in the lungs of both wild-type and mutant-infected mice (FIG. 8), but largely absent from unconsolidated areas of the lung (not shown). However, the extent of fibrin(ogen) formation in the Δpla infection appeared greater than in the CO92-infected lungs, especially considering the significantly increased numbers of bacteria in the lungs of the wild-type-infected mice by this time (FIG. 1B). In addition, in the wild-type infection the intensity of fibrin(ogen) staining appeared reduced in areas of bacterial microcolonies or large numbers of bacilli, suggesting that there may be alterations in fibrin deposition and/or fibrin clearance in the local vicinity of Y. pestis bacteria. This is consistent with the bacterial cell surface expression of Pla, where plasminogen activation and α₂-antiplasmin inactivation would be greatest.

### REFERENCES

1. R. D. Perry, J. D. Fetherston, Clin. Microbiol. Rev. 10, 35 (1997).

2. T. V. Inglesby et al., J. Amer. Med. Assoc. 283 (2000).

3. G. I. Viboud, J. B. Bliska, Annu. Rev. Microbiol. 59, 69 (2005).

4. X. Z. Huang, M. P. Nikolich, L. E. Lindler, Clin. Med. Res. 4, 189 (2006).

5. L. E. Lindler, in Encyclopedia of medical diagnostics and proteomics J. Fuchs, M. Podda, Eds. (2004) pp. 1-5.

6. T. L. Cover, R. C. Aber, N. Engl. J. Med. 321, 16 (1989).

7. J. Parkhill et al., Nature 413, 523 (2001).

8. O. A. Sodeinde et al., Science 258, 1004 (1992).

9. S. L. Welkos, A. M. Friedlander, K. J. Davis, Microb. Pathog. 23, 211 (1997).

10. S. Welkos et al., Vaccine 20, 2206 (2002).

11. S. V. Samoilova, L. V. Samoilova, I. N. Yezhov, I. G. Drozdov, A. P. Anisimov, J. Med. Microbiol. 45, 440 (1996).

12. J. M. Doll et al., Am. J. Trop. Med. Hyg. 51, 109 (1994).

13. W. W. Lathem, S. D. Crosby, V. L. Miller, W. E. Goldman, Proc. Natl. Acad. Sci. U.S.A. 102, 17786 (2005).

14. F. Sebbane, D. Gardner, D. Long, B. B. Gowen, B. J. Hinnebusch, Am. J. Pathol. 166, 1427 (2005).

15. S. A. Handley, P. H. Dube, V. L. Miller, Proc. Natl. Acad. Sci. U.S.A. (2006).

16. J. E. Celis, P. Madsen, A. Celis, H. V. Nielsen, B. Gesser, FEBS Lett. 220, 1 (1987).

17. R. Lutz, H. Bujard, Nucleic Acids Res. 25, 1203 (1997).

18. M. Kukkonen et al., Mol. Microbiol. 40, 1097 (2001).

19. O. A. Sodeinde, J. D. Goguen, Infect. Immun. 57, 1517 (1989).

20. I. K. Mullarky et al., Infect. Immun. 73, 3888 (2005).

21. L. L. Johnson, K. N. Berggren, F. M. Szaba, W. Chen, S. T. Smiley, J. Exp. Med. 197, 801 (2003).

22. M. J. Flick et al., J. Clin. Invest. 113, 1596 (2004).

23. Applied Biosystems, in ABI Prism 7700 Sequence Detection System User Bulletin #2. (1997).

24. K. A. Datsenko, B. L. Wanner, Proc. Natl. Acad. Sci. U.S.A. 97, 6640 (2000).

25. J. Parkhill et al., Nature 413, 523 (2001).

26. K. H. Choi et al., Nat. Methods 2, 443 (2005).

27. M. Gossen, H. Bujard, Nucleic Acids Res. 21, 4411 (1993).

28. R. Lutz, H. Bujard, Nucleic Acids Res. 25, 1203 (Mar. 15, 1997).

29. W. G. Miller, J. H. Leveau, S. E. Lindow, Mol. Plant Microbe Interact. 13, 1243 (2000).

30. L. C. Huang, E. A. Wood, M. M. Cox, J. Bacteriol. 179, 6076 (1997).

31. F. Qian, W. Pan, Infect. Immun. 70, 2029 (2002).

What is claimed is:

1. A method of decreasing the proliferation of Y. pestis in the lungs of a subject infected with Y. pestis, the method comprising inhibiting the protease activity of Pla.

2. The method of claim 1, wherein decreasing the proliferation of Y. pestis in the lungs delays the onset of the pro-inflammatory stage of pneumonic plague.

3. The method of claim 2, further comprising administering an antibiotic to the subject.

4. The method of claim 1, wherein the Pla protease activity is selected from the group consisting of Pla activation of plasminogen, Pla degradation of α₂-antiplasmin, and a combination thereof.

5. The method of claim 1, wherein inhibiting the protease activity of Pla inhibits the dissolution of fibrin clots in the lungs of the subject.

6. A method of decreasing the proliferation of Y. pestis in the lungs of a subject infected with Y. pestis, the method comprising inhibiting the dissolution of fibrin clots in the lungs of the subject.

7. The method of claim 6, wherein decreasing the proliferation of Y. pestis in the lungs delays the onset of the pro-inflammatory stage of pneumonic plague.

8. The method of claim 7, further comprising administering an antibiotic to the subject.

9. The method of claim 6, wherein the dissolution of fibrin clots is inhibited by inhibiting plasmin activity.

10. The method of claim 9, wherein plasmin activity is inhibited by inhibiting plasminogen activation.

11. The method of claim 10, wherein plasminogen activation is inhibited by inhibiting the protease activity of Pla.

12. The method of claim 9, wherein plasmin activity is inhibited by preventing the degradation of α₂-antiplasmin.

13. The method of claim 12, wherein the degradation of α₂-antiplasmin is prevented by inhibiting the protease activity of Pla.

14. The method of claim 9, wherein plasmin activity is inhibited by a serine protease inhibitor.

15. A method for delaying the onset of the pro-inflammatory stage of pneumonic plaque in a subject, the method comprising inhibiting Pla protease activity.

16. The method of claim 15, further comprising administering an antibiotic to the subject.

17. The method of claim 15, wherein the Pla protease activity is selected from the group consisting of Pla activation of plasminogen, Pla degradation of α₂-antiplasmin, and a combination thereof.

18. The method of claim 15, wherein inhibiting the protease activity of Pla decreases fibrin deposition in the lungs of the subject.

19. The method of claim 15, wherein inhibiting Pla protease activity decreases the proliferation of Y. pestis in the lungs of the subject.


SEQUENCE LISTING (20 sequences)

SEQ ID NO: 1 - DNA, 25 bp, Yersinia pestis
cgcctgctggctgcacttgtcgttg

SEQ ID NO: 2 - DNA, 51 bp, Yersinia pestis
gaagcagctccagcctacaccattagacacccttaatctctctgcatgaac

SEQ ID NO: 3 - DNA, 51 bp, Yersinia pestis
ggtcgacggatccccggaatgaaaaatacagatcatatctctcttttcatc

SEQ ID NO: 4 - DNA, 26 bp, Yersinia pestis
ctggagagcaagtaatgagaacatta

SEQ ID NO: 5 - DNA, 46 bp, Yersinia pestis
gaagcagctccagcctacactcagaagcgatattgcagacccgccg

SEQ ID NO: 6 - DNA, 20 bp, Yersinia pestis
ggccctttcgtcttcacctc

SEQ ID NO: 7 - DNA, 20 bp, Yersinia pestis
tagctcctgaaaatctcgcc

SEQ ID NO: 8 - DNA, 28 bp, Yersinia pestis
gggagctcttagcgcgaattgtcgaggg

SEQ ID NO: 9 - DNA, 29 bp, Yersinia pestis
cgggatcccctggcagtttatggcgggcg

SEQ ID NO: 10 - DNA, 30 bp, Yersinia pestis
aactgcagctttcaccagcgtttctgggtg

SEQ ID NO: 11 - DNA, 23 bp, Yersinia pestis
gggtacctttctcctctttaatg

SEQ ID NO: 12 - DNA, 47 bp, Yersinia pestis
cattaaagaggagaaaggtacccatgagtaaaggagaagaacttttc

SEQ ID NO: 13 - DNA, 32 bp, Yersinia pestis
cgggatccttatttgtatagttcatccatgcc

SEQ ID NO: 14 - DNA, 46 bp, Yersinia pestis
cattaaagaggagaaaggtacccatgaagaaaagttctattgtggc

SEQ ID NO: 15 - DNA, 29 bp, Yersinia pestis
cccccgggtcagaagcgatattgcagacc

SEQ ID NO: 16 - DNA, 25 bp, Yersinia pestis
gcgccccgtcattatggtgaaaaag

SEQ ID NO: 17 - DNA, 26 bp, Yersinia pestis
acagatcacgcatctcatcctgctac

SEQ ID NO: 18 - DNA, 27 bp, Yersinia pestis
gtagcaggatgagatgcgtgatctgtc

SEQ ID NO: 19 - DNA, 27 bp, Yersinia pestis
gcacatgataatgctgagcactatatg

SEQ ID NO: 20 - DNA, 27 bp, Yersinia pestis
catatagtgctcagcattatcatgtgc